(19) United States
(12) Patent Application Publication
(10) Pub. No.: US 2006/0173213 A1
(43) Pub. Date: Aug. 3, 2006

(54) Nucleophilic acyl substitutions of anhydrides catalyzed by oxometallic complexes

(76) Inventor: Chien-Tien Chen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/339,623

(22) Filed: Jan. 26, 2006

(30) Foreign Application Priority Data
Jan. 28, 2005 (TW) 094102821

(51) Int. Cl. C07C 53/00

(52) U.S. Cl. 562/887

(57) ABSTRACT

The present invention discloses a method of nucleophilic acyl substitution (NAS) of anhydrides catalyzed by oxometalic complex. According to the mentioned method, NAS reaction between anhydride(s) and highly functionalized protic nucleophile can be catalyzed by oxometallic complexes, wherein the oxometallic complexes compris the metals selected from IVB, VB, and VIB groups.

### BACKGROUND OF THE INVENTION

1. Field of the Invention

The present invention is generally related to nucleophilic acyl substitutions (NAS) catalyzed by oxometallic complexes, and more particularly to nucleophilic acyl substitutions of anhydrides catalyzed by oxometallic complexes.

2. Description of the Prior Art

The acylation of nucleophilic reagents, such as alcohols, amines and thiols, is an important topic in organic synthesis, particularly in functional group transformations. The resultant products of acylations can be esters, amides and thioesters, which represent three major categories of acid derivatives. These three classes constitute important functional group componets or key intermediates in organic synthesis and biochemistry.

In the past five years, trimethylsilyl trifluoromethanesulfonates (TMS triflate), triflates or perchlorates derived from metal salts have been widely employed as catalysts in nucleophilic acyl substitution (NAS) reactions of anhydrides by alcohols with satisfactory reactivity. However, in the case of the nucleophilic reagents bearing acid-sensitive functional groups, such as acetonide, tetrahydrofuranyl ether (THP ether), allyl group, stilbene-type diol, functional group compatibility issue remains to be solved. Due to the high reactivity and moisture sensitivity associated with TMS triflates, NAS reactions often need to be operated at or below 0° C. in order to reduce damages to other existing functional groups. Thus, it results in more sophisticated or inconvenient operation of the catalytic reaction. Moreover, these metal triflates are often obtained by direct mixing of metal oxides with excess amount of hot triflic acid (trifluoromethane sulfonic acid) or by mixing metal halides with silver triflates. Residue of triflic acid or silver triflate may interfere with or result in over reactivity in the catalytic process due to their intrinsic reactivity.

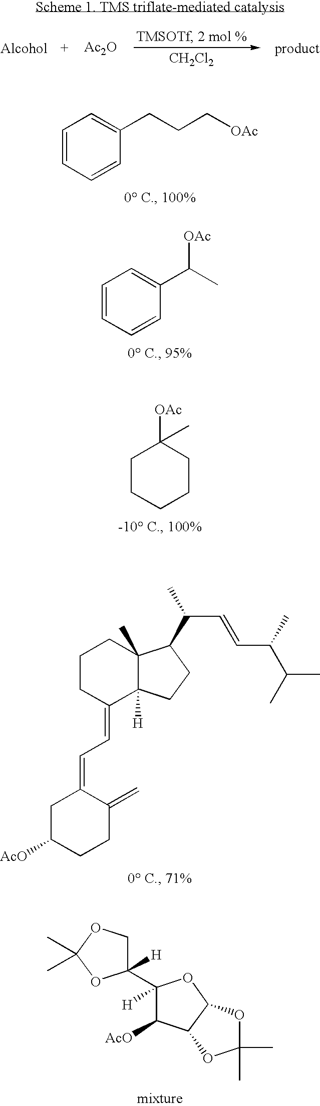

In the recent years, NAS reactions catalyzed by metal triflates constitute popular research topics. Some representative examples of metal triflates are shown in Table 1. They have diaplayed satisfactory reactivity towards NAS reactions. In addition, it can be applied to NAS by phenols, amines, thiols, and alcohols. However, they were subsequently verified to be the precatalysts of HOTf, HClO₄, CH₃C(O)OTf during NAS reactions.

TABLE 1
Metal triflates and perchlorates catalyzed acetylation
reactions

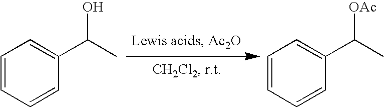

| Lewis acids (1-10 mol %) | time (h) | yield (%) |
|---|---|---|
| LiOTf | 17 | 96 |
| Bi(OTf)₃ | 2 | 95 |
| In(OTf₃) | 0.5 | 98 |
| Sc(OTf)₃ | 1 | 95 |
| Cu(OTf)₂ | 1 | 92 |
| Sn(OTf)₂ | 1 | 90 |
| Mg(ClO₄)₂ | 0.25 | 95 |
| BiO(ClO₄) | 0.2 | 90 |

NAS reactions catalyzed by metal triflates have become popular and useful in the recent years. Notably, such reaction systems still encounter many limitations, such as over reactivity, requirement of lower temperature to suppress side reactions, operation inconvenience due to moisture sensitivity of metal triflates, damages to acid-sensitive functional groups, high cost in catalyst operation, and compatibility with functional groups, etc. In the last case, for instance, the NAS by allyl alcohols catalyzed by Sc(OTf)₃ normally led to rearranged by-products. Besides, in the case of disulfide compounds, the disulfide bond is destroyed. Moreover, in the case of Ce(OTf)₃ during NAS process, tertiary alcohols led to extensive elimination products. Another limitation is that the chemo-selectivity between primary alcohols and phenols is very poor.

It has been reported that NAS reactions catalyzed by metal perchlorates, such as Mg(ClO₄)₂, LiClO₄ and BiO(ClO₄), also showed promising results.

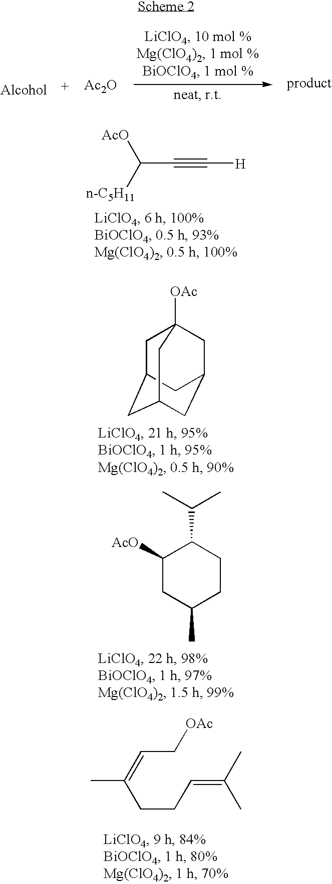

However, it should be noted that perchlorates are potential explosives at elevated temperature. Special cares during synthesis and handling need to be done due to existing danger. Therefore, such catalytic system is not suitable for industry application regardless of the operating role of HClO₄.

In view of the previous researches and reports mentioned above, it is important to develop a more advanced, new catalytic, neutral, and easily operative protocol towards NAS reactions between anhydrides and nucleophilic reagents. Furthermore, applying a new NAS strategy to compounds bearing either acid-sensitive or base-sensitive functional groups with high chemical yields and chemo-selectivity remains in great demand and a main focus in chemical and pharmaceutical industry.

### SUMMARY OF THE INVENTION

In view of the aforementioned invention background and to further fulfill the requirements from the industry, the present invention provides a new and more advanced method for NAS of anhydrides by protic nucleophiles catalyzed by water tolerant oxometallic complexes.

One major objective of the present invention is to provide a handy and highly reliable method for NAS of anhydrides by various functionalized protic nucleophiles catalyzed by oxometallic complexes. According to the present invention, the new catalytic protocol is readily operable in large scale, highly water tolerant, high chemo-selectivity, and excellent yielding. Therefore, the present invention has valuable economic advantages for industrial applications. Furthermore, the above oxometallic complexes can be recycled from the aqueous layer after workup and remain catalytically active for at leat 5 consecutive runs. Therefore, the method according to the present invention is also environmentally benign.

Another objective of the present invention is to provide an unprecedented and reliable method for NAS of in-situ-generated anhydrides with protic nucleophiles catalyzed by oxometalic complexes.

According to the above objectives, the present invention discloses a method for NAS of anhydrides catalyzed by oxometallic complexes. The method uses oxometalic complexes to catalyze the NAS reactions between anhydrides and nucleophilic reagents wherein the metals of the oxometallic complexes are selected from group IV-B, V-B and VI-B elements.

### DESCRIPTION OF THE PREFERRED EMBODIMENTS

What is probed into the invention is a method for NAS of anhydrides catalyzed by oxometallic complexes. Detail descriptions of the representative catalytic protocol, catalyst structures, and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limitation of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressively not limited except those specified in the accompanying claims.

In a first embodiment of the present invention, a method for NAS of anhydrides catalyzed by oxometallic complexes is disclosed. The general equation for the reaction of the method is shown in Scheme 3. R¹ and R² can be the same or different and comprise any one selected from the group consisting of cyclic aliphatic, acyclic aliphatic, aromatic, and heterocyclic moiety. R³ in the nucleophilic reagent-R³YH comprises any one selected from the group consisting of cyclic aliphatic, acyclic aliphatic, aromatic, and heterocyclic moiety. R³ further comprises at least one selected from the group consisting of alkene moiety, ether moiety, ester moiety, lactone moiety, allylic moiety, aldehyde moiety, ketone moiety, acetonide moiety, imide moiety, amide moiety, carbohydrate moiety, peptide moiety, disulfide moiety, and other functional groups known by one skilled in the art. R³ further comprises a solid-state carrier, such as general solid-state resins and solid-state nanocarriers. Y in the nucleophilic reagent-R³YH comprises any one selected from the group consisting of O, NH, and S. The metal in the oxometalic complex comprises any one selected from group IVB, VB and VIB transition metal elements.

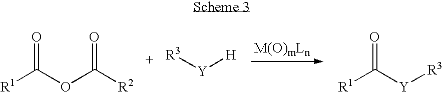

In a preferred example of this embodiment, the metal M is group IVB transition metal; m=1; and, said Lₙ is selected from the following group: 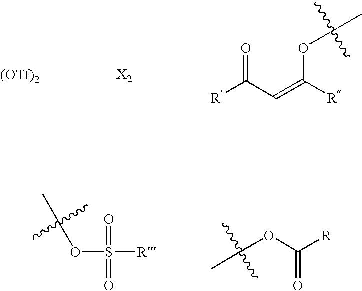
wherein X is halogen element;

R′=R″ or R′≠R″;

R′, R″ comprise alkyl, aryl, or N, O, P or S-containing heterocyclic group;

R′″ comprises alkyl, aryl, or N, O, P or S-containing heterocyclic group; and,

R comprises alkyl, aryl, or N, O, P or S-containing heterocyclic group.
In this example, after the NAS reaction is proceeded for about 0.3-168 hours, the product yield of the reaction is in a range of 40-100%. For further demonstration of the example, a preferred reaction of the example is shown as the following.

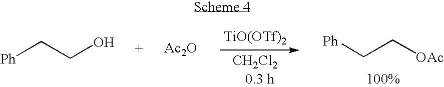

In another preferred example of this embodiment, the metal M is group VB transition metal; m=1; and, Lₙ is selected from the following group: (OTf)₂(THF)₂, Cl₂(THF)₂, (OAc)₂(THF)₂, (OTs)₂, (OSO₂C₁₂H₂₅)₂, (SO₃-alkyl)₂, (SO₃-alkyl)₂(THF)₂ or other moiety known by one skilled in the art. In this example, after the NAS reactions are proceeded for 9˜76 hours, the product yields of the reaction are about 60˜90%. For further demonstration of the example, a preferred reaction of the example is shown as the following:

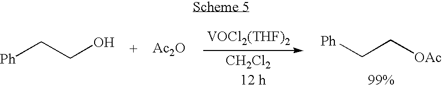

In another preferred example of this embodiment, the metal M is group VIB transition metal element. When m=1, Lₙ can be X₄ or other moiety known by one skilled in the art wherein X is halogen element. When m=2, Lₙ comprises one moiety selected from the following group or other moiety known by one skilled in the art:

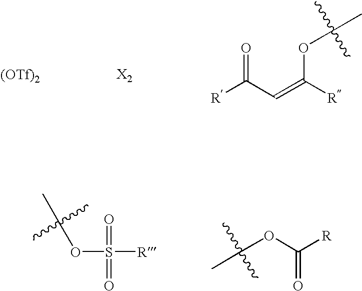

wherein X is halogen element;

R′=R″ or R′≠R″;

R′, R″ comprise alky, aryl, or N, O, P or S-containing heterocyclic group;

R′″ comprises alkyl, aryl, or N, O, P or S-containing heterocyclic group; and,
R comprises alkyl, aryl, or N, O, P or S-containing heterocyclic group. In this example, after the NAS reactions are proceeded for 0.1˜51 hours, the product yields of the reaction are greater than 95%. For further demonstration of the example, a preferred reaction of the example is shown as the following.

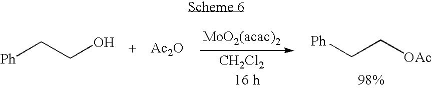

### EXAMPLE

Process of the NAS Reaction:

In a dry 50-mL, two-necked, round-bottomed flask was placed an oxometallic complex (0.01 mmol) in 3 mL of anhydrous solvent (CH₂Cl₂ was used here.) under nitrogen atmosphere. Then, a given anhydride (1.5 mmol) was added to this solution at room temperature. The mixture of the catalyst and anhydride was stirred for 30 minutes at room temperature. The nucleophilic reagent (1.0 mmol dissolved in 2 mL of anhydrous CH₂Cl₂) was added dropwise into the mixture of the catalyst and anhydride. After completion of the reaction as monitored by TLC, the reaction was quenched by cold, saturated aqueous NaHCO₃ solution (5 mL). The resulting separated organic layer was dried by MgSO₄ powders, filtered, and evaporated by a rotary evaporator to remove excess solvent. In general, the crude product with satisfactory high purity was obtained. It is not required to use column chromatography to purify the crude product.

Process for the Recycling of Catalyst:

In a dry 50-mL, two-necked, round bottomed flask was placed an oxometallic complex (0.5 mmol) in 50 mL of anhydrous solvent (such as CH₂Cl₂) under nitrogen atmosphere. Then, anhydride (75 mmol) was added to this solution at room temperature. The mixture of the catalyst and anhydride was stirred for 30 minutes at room temperature. The nucleophilic reagent (50 mmol dissolved in 20 mL of anhydrous CH₂Cl₂) was added dropwise into the mixture of the catalyst and anhydride. After completion of the reaction as monitored by TLC, the reaction was quenched by ice water (100 mL). The excess amount of water was removed from the resulting separated water layer by a rotary evaporator. Then, it was dried by a vacuum pump for 2 hours to obtain the recycled oxometallic complex (recovery yield>95%).

### 2-Phenylethyl Acetate

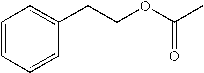

Data: ¹H NMR (200 MHz, CDCl₃) 7.32-7.20 (m, 5H), (4.28 (t, J=7.2, 2H), 2.93 (t, J=7.2, 2H), 2.03 (s, 3H);

¹³C NMR (50 MHz, CDCl₃) 171.07, 137.84, 128.89, 128.51, 126.57, 64.85, 34.99, 20.83; TLC R_{f}0.62 (EtOAc/hexane, 1/20).

In another embodiment of the present invention, a method for NAS of in-situ-generated mixed anhydrides catalyzed by oxometallic complexes is disclosed. The reaction equation in the method is shown in Scheme 7. R⁴ and R⁵ are either the same or different and comprise any one selected from the group consisting of cyclic aliphatic, tertiary alkoxy, aromatic, heterocyclic, and sterically demanding alkane moiety such as iso-butyl moiety or tert-butyl moiety. R⁶ in the carboxylic acid reagent comprises any one selected from the group consisting of cyclic aliphatic, acyclic aliphatic, aromatic, and heterocyclic moiety. R⁶ further comprises at least one selected from the group consisting of alkene, ether, ester, lactone, acrylate, aldehyde, ketone, acetonide, imide, amide, carbohydrate, peptide, and disulfide moiety, and other functional groups known by one skilled in the art. R⁶ further comprises a solid-state carrier, such as general solid-state resin and solid-state nanocarrier.

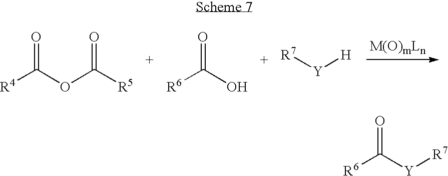

R⁷ in the nucleophilic reagent R⁷YH comprises any one selected from the group consisting of cyclic aliphatic, acyclic aliphatic, aromatic, and heterocyclic moiety. R⁷ further comprises at least one selected from the group consisting of alkene, ether, ester, lactone, acrylate, aldehyde, ketone, acetonide, imide, amide, carbohydrate, peptide, and disulfide moiety, and other functional groups known by one skilled in the art. R⁷ further comprises a solid-state carrier, such as general solid-state resin and solid-state nanocarrier. Y in the nucleophilic reagent-R⁷YH comprises one selected from the group consisting of O, NH and S. The metal of the oxometalic complex comprises any one selected from the group consisting of group IVB, VB and VIB transition metal element.

In a preferred example of this embodiment, the metal M is group IVB transition metal; m=1; and, said Lₙ is selected from the following group: 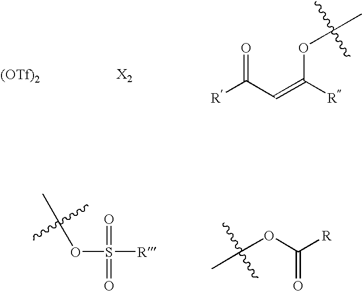
wherein X is halogen element;

R′=R″ or R′≠R″;

R′, R″ comprise alky, aryl, or N, O, P or S-containing heterocyclic group;

R′″ comprises alky, aryl, or N, O, P or S-containing heterocyclic group; and,

R comprises alkyl, aryl, or N, O, P or S-containing heterocyclic group.

In another preferred example of this embodiment, the metal M is group VB transition metal; m=1; and, Lₙ is selected from the following group: (OTf)₂(THF)₂, Cl₂(THF)₂, (OAc)₂(THF)₂, (OTs)₂, (OSO₂C₁₂H₂₅)₂, (SO₃-alkyl)₂, (SO₃-alkyl)₂(THF)₂ or other moiety known by one skilled in the art.

In another preferred example of this embodiment, the metal M is group VIB transition metal element. When m=1, Lₙ can be X₄ or other moiety known by one skilled in the art wherein X is halogen element. When m=2, Lₙ comprises one moiety selected from the following group or other moiety known by one skilled in the art:

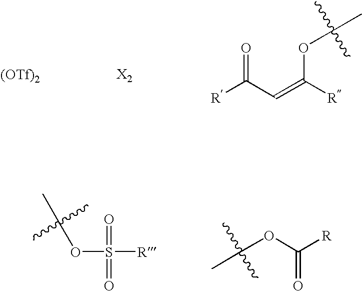

wherein X is halogen element;

R′=R″ or R′≠R″;

R′, R″ comprise alkyl, aryl, or N, O, P or S-containing heterocyclic group;

R′″ comprises alkyl, aryl, or N, O, P or S-containing heterocyclic group; and,
R comprises alkyl, aryl, or N, O, P or S-containing heterocyclic group. For further demonstration of the example, a preferred reaction of the example is shown as the following.

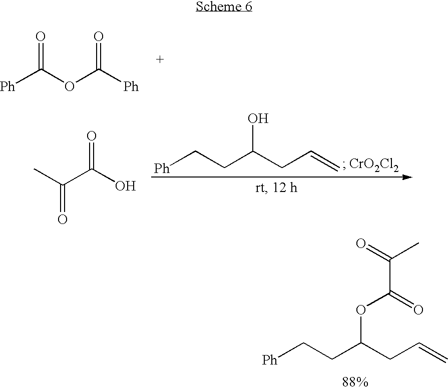

### EXAMPLE

In a dry 50-mL, two-necked, round bottomed flask was placed an oxometalic complex (0.05 mmol) in 2 mL of anhydrous solvent (CH₂Cl₂ was used here.) were under nitrogen atmosphere. Then, carboxylic acid (1.1 mmol) was added into a given anhydride (1.1 mmol) at room temperature. The mixture of the oxometalic complex, carboxylic acid, and anhydride was stirred for 0.5-2 hours at room temperature to form a mixed anhydride-oxometallic species adduct. The nucleophilic reagent (1.0 mmol dissolved in 5 mL of anhydrous CH₂Cl₂) was added dropwise into the anhydride mixture. After completion of the reaction as monitored by TLC, the reaction was quenched by cold, saturated aqueous NaHCO₃ solution (20 mL). The resulting separated organic layer was dried by MgSO₄ powders, filtered, and evaporated by a rotary evaporator to remove excess solvent. The crude product was purified by column chromatography (EtOAc/hexane, 3/97) to obtain the product.

### 2-Oxo-propionic acid 1-phenethyl-but-3-enyl ester

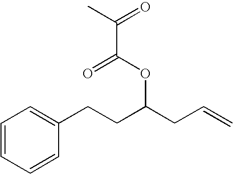

Data: ¹H NMR (400 MHz, CDCl₃) 7.30-7.15 (m, 5H), 5.79-5.69 (m, 1H), 5.13-5.05 (m, 3H), 2.72-2.60 (m, 2H), 2.45-2.41 (m, 5H), 2.12-1.95 (m, 2H); ¹³C NMR (100 MHz, CDCl₃) 191.97, 160.54, 140.92, 132.72, 128.50, 128.30, 126.13, 118.57, 75.77, 38.50, 34.97, 31.67, 26.74; IR (CH₂Cl₂) 3491 (m), 3052 (s), 2685 (s), 2524 (s), 2306 (s), 1738 (s), 1694 (s), 1605 (m), 1585 (m), 1420 (s), 1319 (s), 1287 (s), 1250 (s), 1177 (m), 1071 (m), 1026 (m), 896 (s); MS (70 eV) 246 (M⁺, 2), 205 (13), 158 (24), 133 (13), 117 (100), 104 (21), 91 (69); TLC R_{f} 0.31 (EtOAc/hexane, 1/9); HR-MS Calcd. For M⁺, C₁₅H₁₈O₃: 246.1256, found: 246.1256.

According to the above, this embodiment discloses a new method to prepare esters, amides or other carboxylic acid derivatives by NAS reactions of in-situ-generated mixed anhydride. In the prior art, heating or even severe reaction condition is required to drive the reaction in order to have less satisfactory or similar product yields as that of this embodiment. There are many reported cases in the prior art that are difficult to operate and have low reaction product yields. However, according to the method of this embodiment, this kind of reaction can be operated under very mild reaction condition. In most situations, the reaction can take place at room temperature with good to excellent product yields.

To sum up, the present invention discloses a method for NAS of anhydrides catalyzed by oxometallic complexes. The NAS reactions, catalyzed by these complexes including group IVB, VB, or VIB transition metal element, exhibit high water tolerant, high chemo-selectivity, and excellent product yields. Furthermore, the above oxometallic complexes have high stability to air and moisture and also can be recycled. On the other hand, the present invention discloses a method for NAS reactions of in-situ-generated anhydrides catalyzed by oxometallic complexes. This method allows for the preparation of highly functionalized carboxylic acid derivatives under milder reaction conditions than those in the prior art. These target acid derivatives cannot be prepared by metal triflate-mediated catalysis described in the prior art. The present invention can be applied to the synthesis of products in various fields, such as biochemistry, medicine, and optical materials, etc.

Obviously many modifications and variations are possible in light of the above representative teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A method for NAS of anhydrides catalyzed by oxometallic complexes, comprising:
providing an anhydride with the structure of 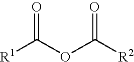
wherein R¹ and R² are either the same or different and comprise any one selected from the group consisting of cyclic aliphatic, acyclic aliphatic, aromatic, and N, O, P or S-containing heterocyclic moiety; and
catalyzing a NAS reaction of said anhydride with a nucleophilic reagent-R³YH by an oxometalic complex wherein R³ comprises any one selected from the group consisting of cyclic aliphatic, acyclic aliphatic, aromatic, and N, O, P or S-containing heterocyclic moiety and Y comprises any one selected from the group consisting of O, NH and S;
wherein said oxometallic complex has the formula as MOₘLₙ;
said nucleophilic reagent has the formula as R³YH; and, said NAS reaction has the following general chemical equation: 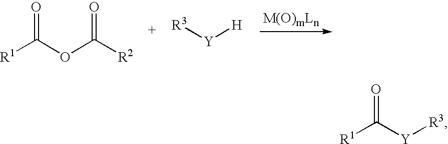
wherein said metal M of said oxometallic complex comprises group IVB or VIB transition metal, and m and n are integers greater than or equal to 1.

2. The method according to claim 1, wherein said R³ further comprises at least one selected from the group consisting of alkene, ether, ester, lactone, acrylate, aldehyde, ketone, acetonide, imide, amide, carbohydrate, peptide, and disulfide moiety.

3. The method according to claim 1, wherein said R³ further comprises a solid-state carrier.

4. The method according to claim 1, wherein said metal M is group IVB transition metal; m=1; and, said Lₙ is selected from the following group: 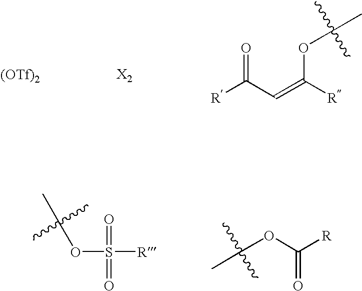
wherein X is halogen element;
R′=R″ or R≠R″;
R′, R″ comprise alkyl, aryl, or N, O, P or S-containing heterocyclic group;
R′″ comprises alkyl, aryl, or N, O, P or S-containing heterocyclic group; and,
R comprises alky, aryl, or N, O, P or S-containing heterocyclic group.

5. The method according to claim 1, wherein said metal M is group VIB transition metal; m=1; and, said Lₙ is X₄ where X is halogen element.

6. The method according to claim 1, wherein said metal M is group VIB transition metal; m=2; and, said Lₙ is selected from the following group: 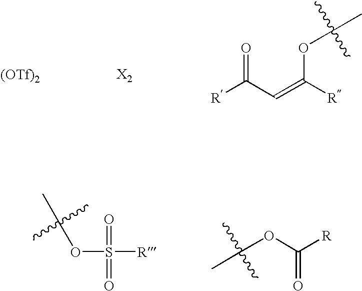
wherein X is halogen element;
R′=R″ or R≠R″;
R′, R″ comprise alkyl, aryl, or N, O, P or S-containing heterocyclic group;
R′″ comprises alkyl, aryl, or N, O, P or S-containing heterocyclic group; and,
R comprises alkyl, aryl, or N, O, P or S-containing heterocyclic group.

7. A method for NAS of anhydrides catalyzed by oxometallic complexes, comprising:
providing an anhydride with structure of 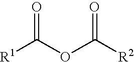
wherein R¹ and R² are either the same or different and comprise any one selected from the group consisting of cyclic aliphatic, acyclic aliphatic, aromatic, and N, O, P or S-containing heterocyclic moiety; and
catalyzing a NAS reaction of said anhydride with a nucleophilic reagent-R³YH by an oxometallic complex wherein R³ comprises any one selected from the group consisting of cyclic aliphati, acyclic aliphatic, aromatic, and N, O, P or S-containing heterocyclic moiety and Y comprises any one selected from the group consisting of O, NH and S;
wherein said metal oxometallic has the formula as MOLₙ; said nucleophilic reagent has the formula as R³YH; and, said NAS reaction has the following general chemical equation: 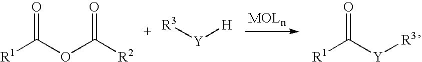
wherein said metal M of said oxometallic complex comprises group VB transition metal and Lₙ comprises one selected from the group consisting of (OTf)₂(THF)₂, Cl₂(THF)₂, (OAc)₂(THF)₂, (SO₃-alkyl)₂ and (SO₃-alkyl)₂(THF)₂.

8. The method according to claim 7, wherein said R³ further comprises at least one selected from the group consisting of alkene, ether, ester, lactone, acrylate, aldehyde ketone, acetonide, imide, amide, carbohydrate, peptide, and disulfide moiety.

9. The method according to claim 7, wherein said R³ further comprises a solid-state carrier.

10. A method for NAS of anhydrides catalyzed by oxometallic complexes, comprising:
providing an anhydride with structure of 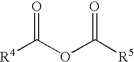
wherein R⁴ and R⁵ are either the same or different and comprise any one selected from the group consisting of cyclic aliphatic, tertiary alkoxy, aromatic moiety, N, O, P or S-containing heterocyclic, iso-butyl, and tert-butyl moiety;
providing a carboxylic acid with structure of 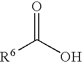
to react with said anhydride so as to form an in-situ-generated anhydride-oxometallic adducut; and
catalyzing a NAS reaction of said anhydride mixture with a nucleophilic reagent-R⁷YH by an oxometallic complex wherein R⁶ and R⁷ are independently selected from the group consisting of cyclic aliphatic, acyclic aliphatic, cyclic aromatic, and N, O, P or S-containing heterocyclic moiety and Y comprises any one selected from the group consisting of O, NH and S;
wherein said oxometallic complex has the formula as MOₘLₙ; said nucleophilic reagent has the formula as R⁷YH; and, said NAS has the following general chemical equation: 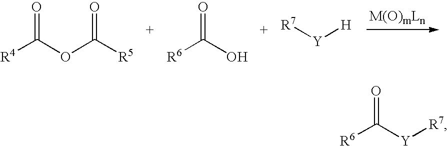
wherein m and n are integers greater than or equal to 1.

11. The method according to claim 10, wherein said R⁶ and R⁷ are independently selected from the group consisting of alkene, ether, ester, lactone, acrylate, aldehyde, ketone, acetonide, imide, amide, carbohydrate, peptide, and disulfide moiety.

12. The method according to claim 10, wherein said R⁶ further comprises a solid-state carrier.

13. The method according to claim 10, wherein said R⁷ further comprises a solid-state carrier.

14. The method according to claim 10, wherein said metal M is group IVB transition metal; m=1; and, said Lₙ is selected from the following group: 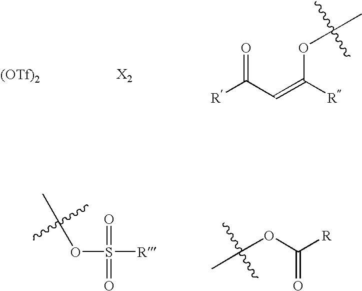
wherein X is halogen element;
R′=R″ or R′≠R″;
R′, R″ comprise alkyl, aryl, or N, O, P or S-containing heterocyclic group;
R′″ comprises alkyl, aryl, or N, O, P or S-containing heterocyclic group; and,
R comprises alkyl, aryl, or N, O, P or S-containing heterocyclic group.

15. The method according to claim 10, wherein said metal M is group VB transition metal; m=1; and, said Lₙ is selected from the group consisting of (OTf)₂(THF)₂, Cl₂(THF)₂, (OAc)₂(THF)₂, (SO₃-alkyl)₂ and (SO₃-alkyl)₂(THF)₂.

16. The method according to claim 10, wherein said metal M is group VIB transition metal; m=1; and, said Lₙ is X₄ where X is halogen element.

17. The method according to claim 10, wherein said metal M is group VIB transition metal; m=2; and, said Lₙ is selected from the following group: 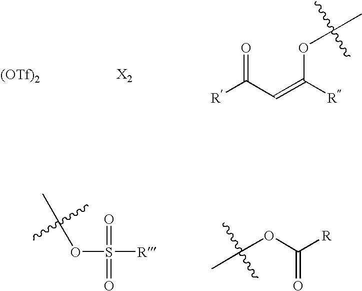
wherein X is halogen element;
R′=R″ or R≠R″;
R′, R″ comprise alkyl, aryl, or N, O, P or S-containing heterocyclic group;
R′″ comprises alkyl, aryl, or N, O, P or S-containing heterocyclic group; and,
R comprises alkyl, aryl, or N, O, P or S-containing heterocyclic group.
